(19) United States
(12) Patent Application Publication
(10) Pub. No.: US 2026/0144455 A1
(43) Pub. Date: May 28, 2026

(54) LEG ISOINERTIAL MEASUREMENT BRACE SYSTEMS AND METHODS

(71) Applicant: UNIVERSITY OF SOUTH FLORIDA, Tampa, FL (US)

(72) Inventors: Aidan Miguel FOY, Tampa, FL (US); Jacob Joseph LOUFEK, Tampa, FL (US); David BOTERO, Tampa, FL (US); Nathan D. SCHILATY, Tampa, FL (US); Benjamin Lewis KEISTER, Tampa, FL (US); Stephanie Lutton CAREY, Tampa, FL (US)

(21) Appl. No.: 19/399,230

(22) Filed: Nov. 24, 2025

Related U.S. Application Data
(60) Provisional application No. 63/724,121, filed on Nov. 22, 2024.
(60) Provisional application No. 63/905,235, filed on Oct. 24, 2025.

(51) Int. Cl. A61B 5/107; A61B 5/00; A61B 90/00

(52) U.S. Cl. CPC A61B 5/1071; A61B 5/0057; A61B 5/4585; A61B 5/6812; A61B 5/742; A61B 5/7475; A61B 90/06; A61B 2090/066

(57) ABSTRACT

A device may include an upper member adapted to fit a wearer's upper leg and a lower member adapted to fit a wearer's lower leg, wherein the upper member and the lower member are rotationally coupled about a rotational axis. A device may include a torque cell operatively coupled to the upper member or the lower member and configured to measure torque at the rotational axis. A device may also include a drive assembly configured to transmit torque to the upper member and the lower member about the rotational axis, and a motor assembly coupled the drive assembly to actuate the drive assembly. A device may also include a controller comprising a non-transitory computer-readable medium storing instructions that, when executed, cause the motor to actuate the drive assembly to deliver a stored torque profile to the upper member and the lower member about the rotational axis.

### CROSS-REFERENCE TO RELATED APPLICATIONS

This Application claims priority to U.S. Provisional Patent Application Ser. No. 63/724,121, filed on Nov. 22, 2024 entitled “LEG ISOINERTIAL MEASUREMENT BRACE (LIMB),” and U.S. Provisional Patent Application Ser. No. 63/905,235, filed on Oct. 24, 2025 entitled “LEG ISOINERTIAL MEASUREMENT BRACE (LIMB),” the entire disclosure of which is incorporated herein by reference.

### BACKGROUND

Devices for assessing isoinertial measurements, limb mechanics, and neuromuscular function may rely on static or constant-load resistance. Some systems may include motors, sensors, or mechanical assemblies to apply a force to a limb and measure the response. In certain applications, variable resistance is provided to better simulate real-world muscle demands. These devices may be used in clinical or research settings to evaluate joint behavior, rehabilitation progress, or muscle performance.

### SUMMARY

In some examples, the techniques described herein relate to a device, including: an upper member adapted to fit a wearer's upper leg and a lower member adapted to fit a wearer's lower leg, where the upper member and the lower member are rotationally coupled about a rotational axis. The device also describes a torque cell operatively coupled to the upper member or the lower member, the torque cell configured to measure torque at the rotational axis. The device also including a drive assembly configured to transmit torque to the upper member and the lower member about the rotational axis, and a motor assembly coupled the drive assembly to actuate the drive assembly. Further, the device describes a controller including a non-transitory computer-readable medium storing instructions that, when executed, cause the motor to actuate the drive assembly to deliver a stored torque profile to the upper member and the lower member about the rotational axis.

In some examples, the techniques described herein relate to a device where the drive assembly includes a capstan drive including a first drum operatively coupled to the motor The drive assembly also including a second drum operatively coupled to the rotational axis, a torque cell, and a cord wrapped around the second drum and fixed to opposing ends of the first drum.

In some examples, the techniques described herein relate to a device, further including at least one angle measurement sensor configured to detect angular displacement at either the motor shaft or the output joint.

In other examples, the techniques described herein relate to a device, further including a first angle measurement sensor disposed at the motor shaft and a second angle measurement sensor disposed at the output joint.

In some examples, the techniques described herein relate to a device, further including an interface configured to receive a torque profile and instructions, and store the received torque profile as the stored torque profile.

In additional aspects, the techniques described herein relate to a device, wherein the torque cell is configured to measure torque at the rotational axis and transmit the measured torque to the controller.

In some examples, the techniques described herein relate to a device, wherein the interface is further configured to output stored torque measurements or received torque measurements to an external computing device or display.

In another example, the techniques described herein relate to a device, wherein the controller is configured to execute a control algorithm including proportional-integral-derivative logic to regulate motor operation based on feedback from the torque cell and angle measurement sensors.

In some examples, the techniques described herein relate to a device, wherein the controller is configured to adjust motor response in real time based on torque measurements received from the torque cell.

In another example, the techniques described herein relate to a device, wherein the controller is further configured to adjust motor response in real time based on a received torque response profile.

In some examples, the techniques described herein relate to a device, wherein the motor assembly is configured to actuate the drive assembly to produce flexion of the lower member relative to the upper member.

In some aspects, the techniques described herein relate to a device, wherein the motor assembly is configured to actuate the drive assembly to produce extension of the lower member relative to the upper member.

### BRIEF DESCRIPTION OF THE DRAWINGS

FIG. 1 illustrates a Leg Isointerial Measurement Brace (LIMB) according to a first example of the present disclosure;

FIG. 2 illustrates another aspect of the LIMB of FIG. 1 positioned on a users leg;

FIG. 3 illustrates the LIMB of FIG. 1 from a front view;

FIG. 4 illustrates the LIMB of FIG. 1 from a side view;

FIG. 5 illustrates the LIMB of FIG. 1 from a rear view;

FIG. 6 illustrates an exploded view of the LIMB of FIG. 1 from an isometric perspective;

FIG. 7 illustrates an isometric view of the motor assembly of the LIMB of FIG. 1;

FIG. 8A illustrates a second example of a LIMB in an unactuated position from a side isometric perspective;

FIG. 8B illustrates the second example of the LIMB of FIG. 8A in an actuated position from a side isometric perspective;

FIG. 9A illustrates an aspect of the second example of FIGS. 8A and 8B from a side view;

FIG. 9B illustrates an aspect of the second example of FIGS. 8A and 8B from a isometric perspective;

FIG. 10 illustrates a third example of a LIMB from a isometric perspective;

FIG. 11 illustrates the motor assembly of the third example of a LIMB as shown in FIG. 10 from an isometric perspective;

FIG. 12 illustrates a fourth example of a LIMB from an isometric perspective;

FIG. 13 illustrates an example control system for use with a LIMB;

FIG. 14A illustrates an example method for the control system of a LIMB;

FIG. 14B illustrates another example control system for use with a LIMB;

FIG. 15 illustrates an example display for use with a LIMB;

FIG. 16 illustrates a second example display for use with the control system and method of a LIMB;

FIG. 17 illustrates a third example display for use with a LIMB;

FIG. 18 illustrates a fourth example display for use with a LIMB, and

FIG. 19 illustrates a fifth example display for use with a LIMB.

### DETAILED DESCRIPTION

Examples of the disclosed technology provide a Leg Isoinertial Measurement Brace (LIMB) for assessing leg muscle neuromotor control for a variety of applications. More specifically, examples of the invention provide an orthopedic device for precise isoinertial torque measurements in rehabilitation and sports medicine environments. The LIMB may include various features and components that optimize muscle strength assessment and isoinertial measurement. Additionally, the LIMB may implement one or more control algorithms that further enhance the accuracy and reliability of torque measurements and muscle evaluations.

The following discussion is presented to enable a person skilled in the art to make and use examples of the disclosure. Various modifications to the illustrated examples will be readily apparent to those skilled in the art, and generic principles presented herein can be applied to other examples and applications without departing from examples of the disclosure. Thus, examples of the disclosure are not intended to be limited to examples shown but are to be accorded the widest scope consistent with the principles and features disclosed herein. The following detailed description is to be read with reference to the figures, in which like elements in different figures have like reference numerals. The figures, which are not necessarily to scale, depict selected examples and are not intended to limit the scope of examples of the disclosure. Skilled artisans will recognize the examples provided herein have many useful alternatives and fall within the scope of examples of the disclosure.

The disclosed leg isoinertial measurement brace (LIMB) is a portable, clinician-operated device designed to deliver controlled torque to a user's leg and measure neuromuscular response under static or quasi-static conditions. Unlike traditional isometric or isokinetic systems, which rely on fixed resistance or constant velocity movement, the LIMB operates in the isoinertial domain—applying a configured torque profile that increases over time while the limb remains at a fixed angle. This allows clinicians to assess how well a patient resists increasing torque without requiring joint movement, simulating realistic muscle loading patterns. Clinicians can select from customizable torque presets, including linear, exponential, or sinusoidal profiles, and run standardized tests across multiple patients. Once activated, the system applies torque through a motorized drive while rotary encoders measure joint angle and torque cells capture real-time force data. As an example, a patient may be instructed to resist the applied torque, and the system logs the response for immediate visualization or later analysis. This approach eliminates examiner bias and enables consistent, repeatable testing in clinical, athletic, or field environments.

The LIMB system may support both flexion and extension testing with bidirectional torque measurement. Its compact form factor allows for sideline use, and the brace can be swapped between legs without recalibration, reducing downtime between tests. The clinician interface enables precise control over test parameters, including torque magnitude, duration, and ramping profile, while emergency stop features ensure patient safety. During testing, the brace remains aligned with the knee's natural axis of rotation, maintaining mechanical concentricity and improving measurement fidelity. The rotary encoders provide high-resolution angle data, enabling dynamic torque-angle profiling and stiffness curve generation. This data can be used to detect neuromuscular instability, track rehabilitation progress, or differentiate between healthy and impaired muscle control. By removing the variability of manual testing and enabling standardized, angle-independent torque delivery, the LIMB introduces a new modality for objective strength assessment and functional diagnostics in orthopedic and sports medicine applications. The system's adaptability and mechanical fidelity make it suitable for both diagnostic and therapeutic applications across diverse operational contexts.

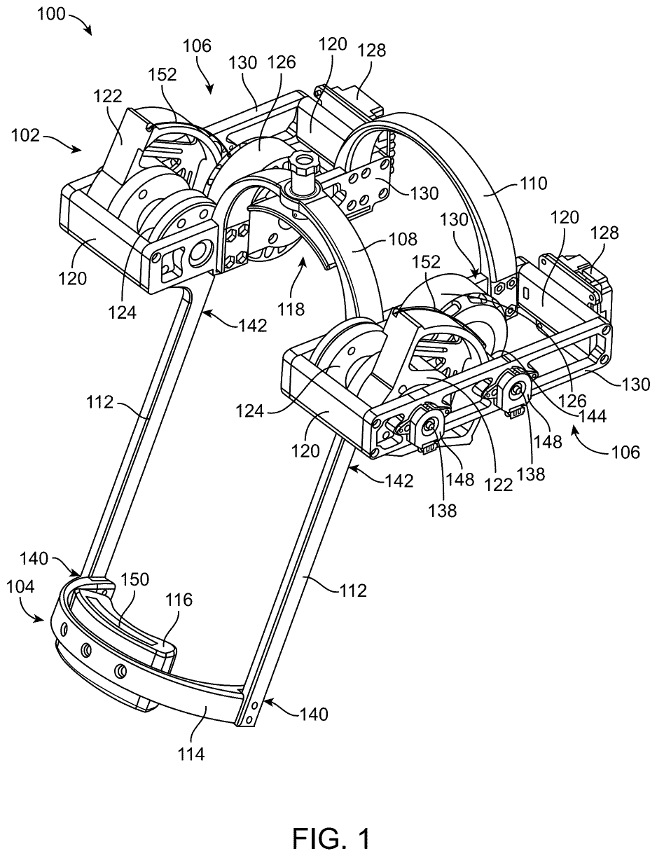

FIG. 1 illustrates an example Leg Isoinertial Measurement Brace, LIMB 100 from a front isometric view in accordance with one or more aspects of the present disclosure. The LIMB 100 may include an upper portion 102 and a lower portion 104. Located about the upper portion 102, the LIMB 100 may include one or more motor assemblies 106, a front bracket 108, and a rear bracket 110, and an adjustment mechanism 118. The lower portion 104 of the LIMB 100 may include one or more extension arms 112 having a first end 142 and a second end 140, and a cross brace 114 with a pad 116. Various components of the LIMB 100 may be constructed from any suitable materials such as composites, polymers, or metals.

Turning to the LIMB 100 of FIG. 1, the upper portion 102 may include one or more motor assemblies 106, the front bracket 108, the rear bracket 110 and the adjustment mechanism 118. The front bracket 108 and the rear bracket 110 may span the upper portion 102 width between the motor assemblies 106. In some examples, the front bracket 108 and the rear bracket 110 may be arched bars that span between two motor assemblies 106. In some examples, the front bracket 108 may be a smaller circular arch than the rear bracket 110 to accommodate the leg of a user. In other cases, the front bracket 108 and rear bracket 110 may be made of a flexible material or a strap 132 as described herein. Within some examples, the brackets may be configured to be adjustable in length or curvature to accommodate a range of user leg sizes and shapes. The brackets may also include padding or a coating to minimize discomfort and reduce slippage during use.

Additionally, the LIMB 100 upper portion 102 may also include an adjustment mechanism 118 located on a bracket such as the front bracket 108. In some cases, the adjustment mechanism 118 may be located on the uppermost arched area of the front bracket 108. It should be known that the adjustment mechanism 118 may be located on one or more brackets or portions of the upper portion 102 of the LIMB 100 such as the rear bracket 110. The adjustment mechanism 118 may comprise a ratcheting assembly, a threaded fastener, a quick-release latch, or any other suitable means for selectively modifying the fit or position of the LIMB 100 relative to the user's leg. The adjustment mechanism 118 may be operable manually or by powered means, such as an electric actuator.

Still referring to FIG. 1, the LIMB 100 may include one or more motor assemblies 106 located on either side of the front bracket 108 and rear bracket 110. It should be known that various combinations and numbers of motor assemblies may be used within the LIMB 100. The motor assembly 106 may include a motor 126, a first drum 122, a second drum 144, a torque cell 124, an attachment member 152, and a controller 128. It should be known that the motor assembly 106 may include one or more motors 126, drums such as drums 122, 144, torque cells 124, and controllers 128 within the assembly. Additionally, the motor assembly 106 of the LIMB 100 may include an end portion 120 and a side portion 130 which may surround a portion of the motor assembly 106. One or more components within the motor assembly 106 may be enclosed partially within the end portion 120 and side portion 130 by coupling means such as shafts, bearings, spacers, and other attachment means known within the art. The motor 106 shown within FIG. 1 may include one or more shafts 138 coupling the components of the motor assembly 106 together. In addition, one or more coupling mechanisms 148 may be used throughout the LIMB 100 and the motor assembly 106.

More specifically, the motor assembly 106 of FIG. 1 is assembled using one or more shafts 138 and one or more coupling mechanisms 148 about the end portion 120 and side portion 130. Additionally, in some examples, the first capstan drum 122 and the second drum 144 may be coupled by attachment member 152. In one example, the attachment member 152 may be a flexible elongate element, such as a rope, cable, belt, or similar tensile member. The attachment member 152 may be arranged such that it wraps around the second drum 144, which may be of a smaller diameter relative to the first capstan drum 122, a plurality of times. For instance, attachment member 152 may be wound in a helical or spiral manner around the circumferential surface of the second drum 144.

Each free end of the rope (attachment member 152) may then be fixedly or removably secured to opposing ends of the first capstan drum 122. In this configuration, the first capstan drum 122 is positioned such that its longitudinal axis is generally parallel to the axis of the second drum 144, although other angular arrangements are contemplated.

The second drum 144 may be operatively connected to a motor 126, such that actuation of the motor 126 causes rotational movement of the second drum 144. As the second drum 144 is rotated by the motor 126, the rope (attachment member 152) winds or unwinds accordingly, thereby exerting a pulling force on one end of the first capstan drum 122 while simultaneously releasing tension from the opposite end. This action causes the first capstan drum 122 to translate or rotate in a reciprocating manner, depending on the overall mechanical arrangement.

In operation, as the motor 126 rotates the second drum 144 in a first direction, one end of the first capstan drum 122 is pulled toward the second drum 144, resulting in a corresponding movement of the first capstan drum 122 in a first direction. Conversely, when the direction of rotation of the second drum 144 is reversed, the opposite end of the first capstan drum 122 is pulled, resulting in movement of the first capstan drum 122 in a second, opposite direction. The wrapping of the rope multiple times around the second drum 144 increases the frictional engagement between the rope and drum, thereby minimizing slippage and enhancing the transfer of motion and force between the two drums.

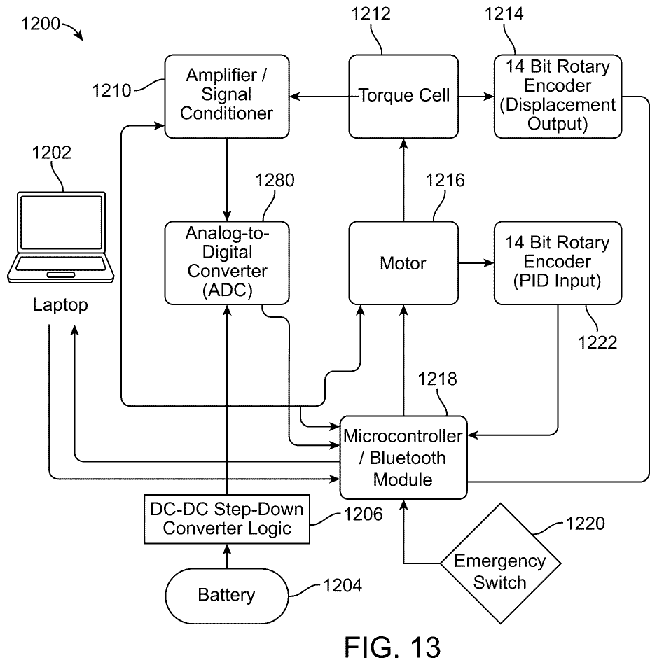

Additionally, various aspects of the controller 128 and operation are described with respect to FIGS. 13-19 The controller 128 may be configured to receive input from sensors and adjust the operation of the motor 126. The torque cell 124 may be arranged to provide continuous feedback on the forces applied during use, enabling dynamic adjustment or data logging. The motor assemblies 106 may be powered by a battery pack or external power source and may include wireless communication modules for remote monitoring or control.

Turning to the lower portion 104 of the LIMB 100, the extension arms 112 having a first end 142 and a second end 140, and a cross brace 114 with a pad 116 are shown. The extension arms 112 may be connected to the upper portion 102 about the first end 142 about the side portion 130. In other examples, the extension arms 112 may be coupled to various parts of the motor assembly 106 such as the torque cell 124 or the motor 126. Within other examples not expressively shown, the extension arms 112 may be telescoping or segmented to allow for adjustment in length to fit different user anatomies. Various attachment combinations of the extension arms 112 should be anticipated. Further, the second end 140 of the extension arms 112 may include the cross brace 114, and the pad 116. The cross brace 114 may be in the shape of an arch which circles a user's leg when in use. Within some examples, the cross brace 114 may be made of a flexible material. The cross brace 114 may include fasteners or hook-and-loop straps to secure the LIMB 100 to the user's leg. The pad 116 may be any type of soft, flexible material capable of supporting a user's leg. The pad 116 may be removable or replaceable to allow for cleaning or customization based on user preference. Pad 116 located on the cross brace 114 may also include mount 150. The mount 150 may be a stiff, rigid material capable of coupling the pad 116 to the cross brace 114. The mount 150 may be configured to permit rotational or translational movement of the pad 116 relative to the cross brace 114 to further enhance comfort and fit.

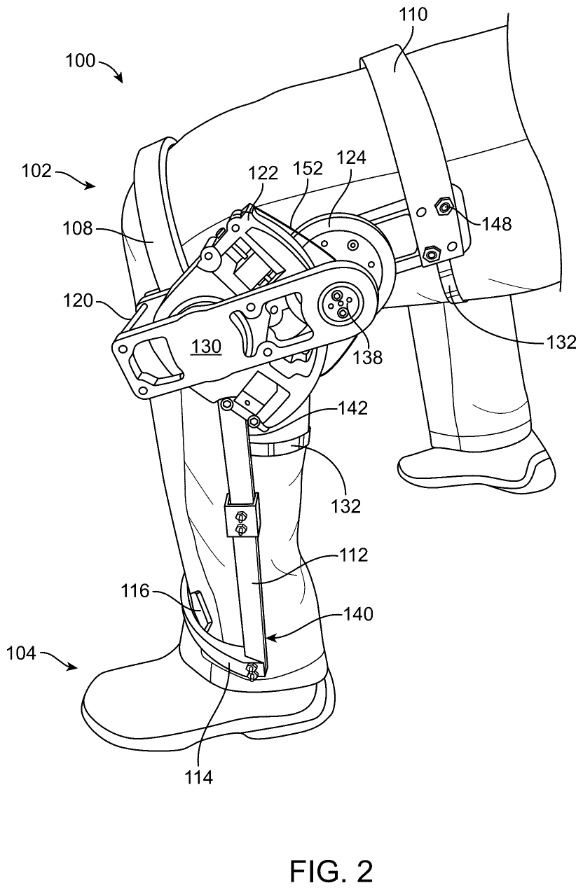

Turning to the elements described with respect to FIG. 2, another aspect of a LIMB 100 is described. Here the LIMB 100 is shown in use on a users leg with the upper portion 102 on the thigh of a user and the lower portion 104 about the users calf. The LIMB 100 may include one or more aspects of the LIMB 100 as described with respect to FIG. 1. The LIMB 100 shown within FIG. 2 may include a strap 132 coupled to the upper portion 102 and the lower portion 104 of the LIMB 100. Specifically, the upper portion 102 may include a strap 132 coupled to the side portion 130 with the strap 132 surrounding a users thigh. Within other examples, the LIMB 100 may also include a strap 132 located about the front bracket 108, rear bracket 110, end portion 120 or a combination thereof. Additionally, the LIMB 100 may include a strap 132 coupled about the extension arms 112 about the lower portion 104, first end 142 second end 140, thus surrounding the users calf. Within other examples, the strap 132 about the lower portion 104 may be coupled to the cross brace 114, pad 116, at least one extension arm 112, or a combination thereof. About the upper portion 102 a strap 132 may be coupled to the side portion 130, end portion 120, front bracket 108, rear bracket 110 or a combination thereof.

Referring to FIG. 2, the LIMB 100 is shown as worn by a user, illustrating the spatial relationship between the upper portion 102 and lower portion 104 when positioned on a leg. In this example, the upper portion 102 is situated about the user's thigh, while the lower portion 104 is disposed about the user's calf. The upper portion 102 is secured to the thigh by one or more straps 132, which may be routed through or about the side portion 130, end portion 120, front bracket 108, and/or rear bracket 110, as depicted. The straps 132 may include adjustment features such as hook-and-loop fasteners, buckles, or ratcheting mechanisms to allow for secure and customizable fitment to a wide range of user anatomies.

Additionally, the motor assembly 106 is shown mounted laterally on the upper portion 102, with the side portion 130 and end portion 120 at least partially enclosing and supporting the motor assembly 106. The first capstan drum 122, second drum 144, and torque cell 124 may be visible, with the attachment member 152 (for example, a rope or cable) operatively connecting the first capstan drum 122 and the second drum 144. The second drum 144 is operatively coupled to the motor 126, allowing for controlled movement of the first capstan drum 122 as described herein. The torque cell 124 may be arranged to measure the force or torque generated during operation, providing feedback to the controller 128, which may be integrated within the motor assembly 106 or disposed elsewhere on the LIMB 100.

As shown within FIG. 2, the extension arms 112 extend downward from the upper portion 102, with a first end 142 coupled to the side portion 130, and a second end 140 coupled to the lower portion 104. The extension arms 112 are shown generally parallel to the user's leg, and may be rigid, telescoping, or segmented to permit adjustment in length as needed to accommodate different users or therapeutic requirements. The lower portion 104 may include the cross brace 114, which may be generally arcuate to conform to the user's shin or calf (e. g anterior or posterior on the user's lower leg), and is provided with a pad 116 to enhance comfort and distribute pressure during use. The cross brace 114 may be secured to the user's leg via an additional strap 132, as shown, which may be routed around the user's calf and fastened.

As further shown in FIG. 2, the LIMB 100 may include a plurality of coupling mechanisms 148, such as bolts, screws, pins, or quick-release fasteners, to enable assembly and disassembly of the various structural and functional components. The coupling mechanisms 148 may also permit selective adjustment or replacement of components, facilitating maintenance and adaptation for different therapeutic protocols or user needs.

One or more sensors may be integrated within the LIMB 100 to detect biomechanical parameters such as position, force, velocity, or acceleration. These sensors may be disposed on or within the extension arms 112, brackets 108, 110, or within the motor assembly 106 itself. Sensor data may be transmitted to the controller 128, which may process the data to adjust the operation of the motor 126, log performance metrics, or provide feedback to the user, clinician, or another operator.

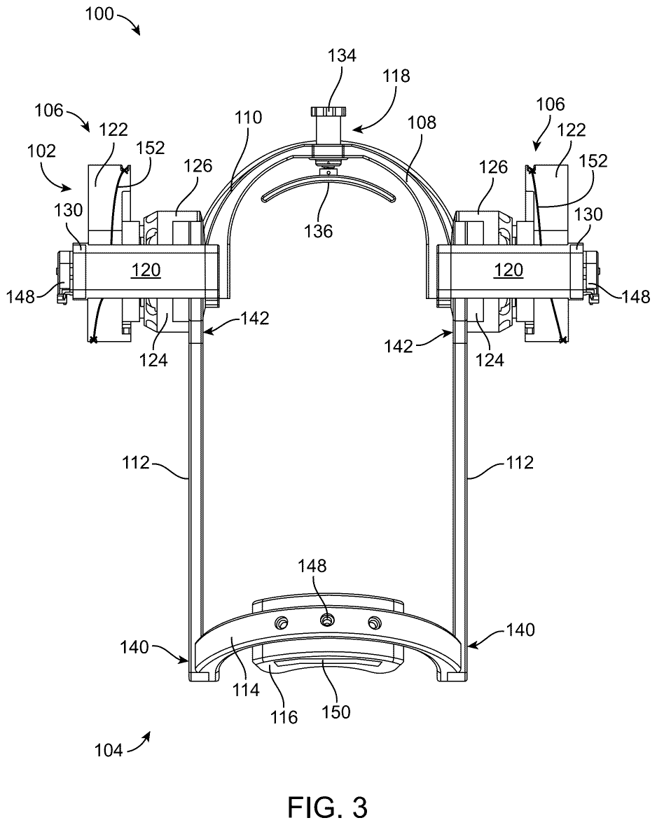

FIG. 3 provides a front view of the LIMB 100., illustrating the upper portion 102 and lower portion 104, the motor assembly 106, end portion 120, side portion 130, motor 126 and the first capstan drum 122. Additionally, the adjustment mechanism 118 is shown on the upper portion 102. Within some examples, the mechanism 118 may include an adjustment knob 134 and a compression member 136. The adjustment knob 134 and the 136 may allow for the customization of fit and alignment to accommodate various user anatomies. The adjustment knob 134 may be operatively coupled to the compression member 136 by a threaded shaft, cam, or similar mechanical interface. Rotation of the adjustment knob 134 causes axial or rotational movement of the compression member 136, thereby increasing or decreasing the compressive force applied to the upper portion 102 of the LIMB 100. The knob 134 may further include tactile or visual indicators, such as detents, markings, or a graduated scale, to provide feedback on the degree of adjustment or compression being applied.

The adjustment mechanism 118 may include a locking feature, such as a spring-loaded detent, ratcheting element, or secondary latch, to prevent unintentional loosening of the adjustment knob 134 during use.

Further, the compression member 136 may be configured as a flexible band, arcuate plate, or padded segment that distributes force evenly across the user's thigh, thus minimizing pressure points and enhancing comfort during prolonged use. In some examples, the compression member 136 may be constructed from materials such as padded polymers, elastomers, or composites, and may include a soft lining to prevent skin irritation.

Still referring to FIG. 3, the front view illustrates the arrangement of the components with respect to a user's extremity, with the upper and lower portions 102, 104 aligning along the extremity. The side portion 130 may be aligned with at least one motor 126 and further enclose other elements of the motor assembly, while also providing attachment points for straps or other securing mechanisms. The arrangement of the extension arms 112, cross brace 114, and pad 116 ensures that the device can be comfortably and securely affixed to the user's limb, while the motor assembly components may be enclosed within end portion 120 and side portion 130.

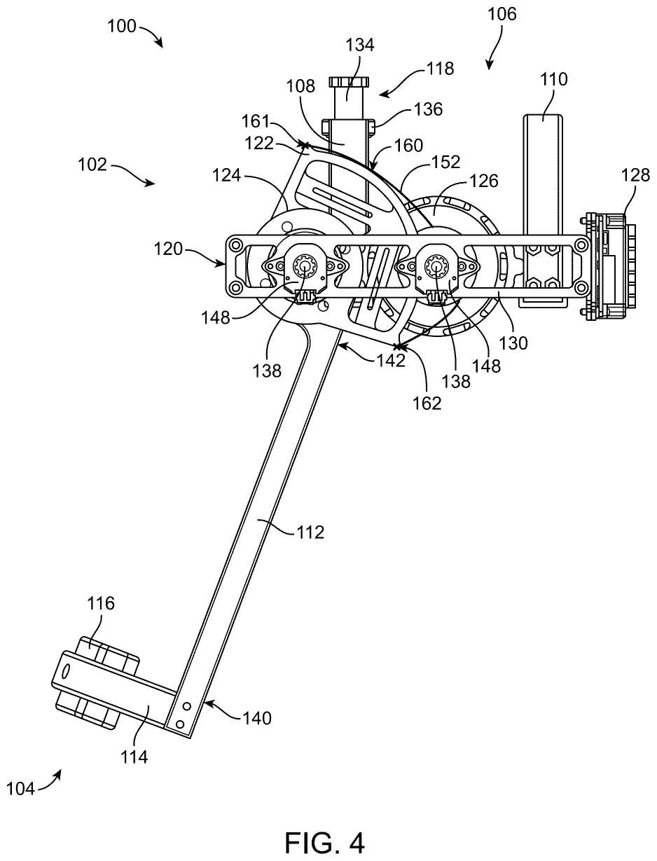

FIG. 4 illustrates the LIMB 100 according to another aspect from a side view. As shown in FIG. 4, the interrelation between the first capstan drum 122, the second drum 144, motor 126, attachment member 152, and torque cell 124 is depicted, as well as the integration with the end portion 120 and side portion 130. Additionally, the LIMB 100 may include a plurality of shafts 138 and one or more coupling mechanisms 148 coupling the components of the motor assembly 106, thus supporting various components. Specifically, the shafts 138 may extend through and connect the first capstan drum 122, motor 126, and torque cell 124 to both the end portion 120 and the side portion 130. In some examples, the one or more coupling mechanisms 148 and shafts 138 may anchoring components to the end portion 120 and side portion 130. Specifically, the shafts 138 may allow for the transmission of torque and rotational motion from the motor 126 to the first capstan drum 122 and second drum 144 while also enabling accurate measurement of force and movement via the torque cell 124. Within additional aspects, the end portion 120 and side portion 130 may serve as rigid mounting points for the shafts 138, enclosing and protecting the sensitive components of the motor assembly 106 while maintaining their spatial orientation. The arrangement of the motor assembly 106 may minimize mechanical play and vibration, thereby increasing the accuracy and reliability of the LIMB 100 during operation.

Additionally, FIG. 4 illustrates the LIMB 100 with the extension arm 112 shown extending downward from the motor assembly 106, terminating at the cross brace 114 and pad 116, which provide lower limb support. As shown, the extension arms 112 may be positioned at an angle relative to the motor assembly 106. Within other aspects, the angle of the extension arms 112 relative to the upper portion 102, end portion 120, front bracket 108, or a combination thereof may be anticipated by the present disclosure. The extension arms 112 may be rigid, telescoping, or segmented, as previously described, to accommodate a range of user sizes and anatomical requirements. The cross brace 114 and pad 116 are positioned to interface with the user's lower limb, ensuring secure attachment and comfort during operation.

Variations in the arrangement, number, and configuration of the components described herein are within the scope of the present disclosure. For example, additional motor assemblies 106 may be provided for bilateral or multi-joint applications, or alternative attachment mechanisms may be employed for securing the LIMB 100 to the user's leg. The LIMB 100 may also be adapted for use on other limbs or joints, such as the arm, or for integration with external support structures or robotic systems. Additionally, the LIMB 100 may be adapted for use with non-human animals, such as horses, dogs, etc.

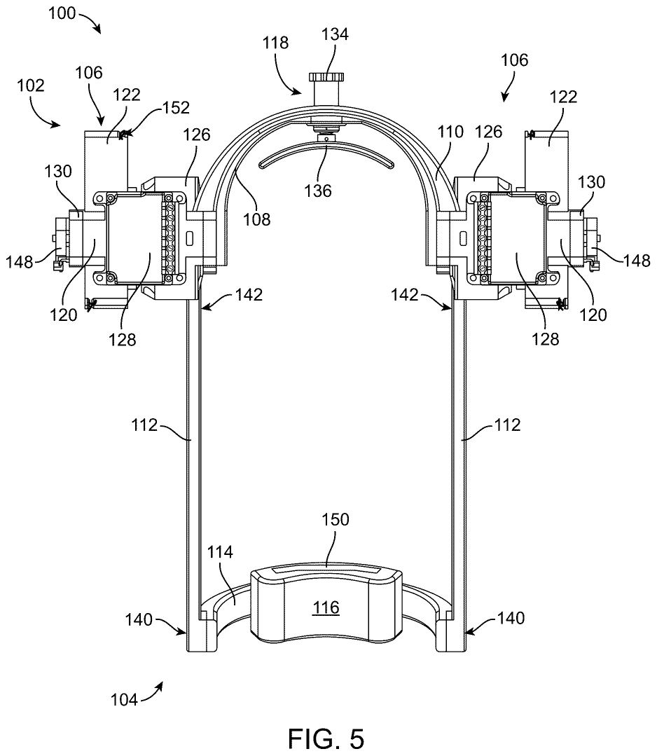

FIG. 5 illustrates an additional example of the LIMB 100 from a rear view. From the rear perspective, the controller 128 is shown mounted to the end portion 120 at the upper portion 102 of the LIMB 100. The controller 128 may be responsible for receiving sensor data, processing control algorithms, and regulating the operation of the motor 126 and associated components. Additional aspects of the controller 128 are described with respect to FIGS. 13-19.

In addition, the controller 128 may be responsible for managing and coordinating the various functions of the LIMB 100. In one or more examples, the controller 128 may comprise a microprocessor or microcontroller-based unit equipped with embedded firmware or software specifically designed for isoinertial measurement and control applications. The controller 128 may receive input signals from various sensors integrated within the LIMB 100, such as the torque cell 124, position encoders, or other force and motion sensors. Controller 128 may process these signals in real time to monitor the operational status of the device, calculate performance metrics, and ensure safe and effective operation.

In addition to sensor data acquisition, the controller 128 may be configured to generate control signals for the motor 126, regulating parameters such as torque, speed, and direction according to predefined protocols or user-selected programs. The controller 128 may also implement safety features such as overload protection, emergency stop, and error detection to prevent device malfunction and ensure user safety. Controller 128 may include communication interfaces, such as USB, Bluetooth, or Wi-Fi, enabling data transfer to external devices for monitoring, logging, or further analysis. In some examples, the controller 128 may be equipped with a user interface, such as buttons, indicator lights, or a display screen, to provide feedback and allow for direct user input or configuration.

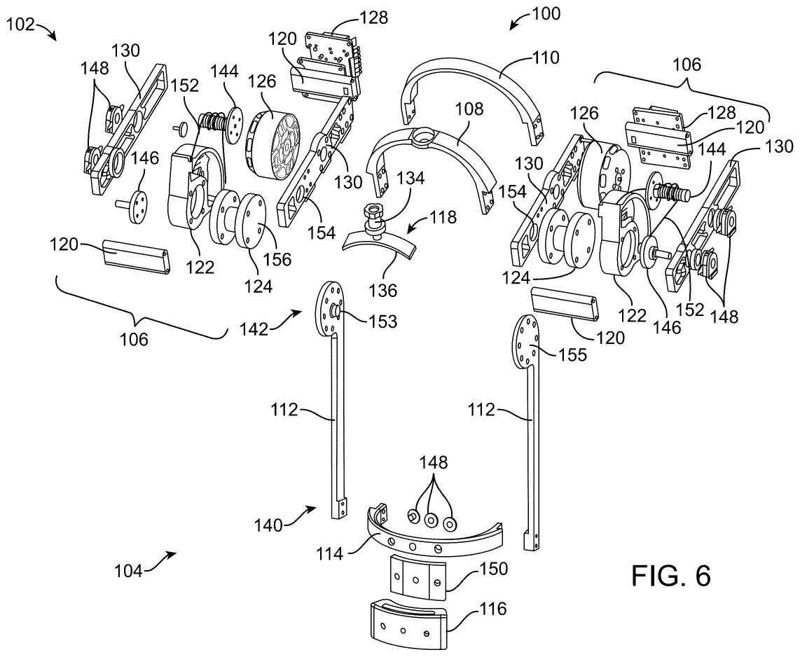

FIG. 6 illustrates an exploded view of the LIMB 100 according to the examples explained above. As shown, the exploded view provides a detailed depiction of the individual components and their spatial relationships prior to assembly. As shown and described herein, the upper portion 102 includes the front bracket 108, rear bracket 110, and the adjustment mechanism 118, which comprises the adjustment knob 134 and compression member 136.

The exploded view further details the construction of the motor assembly 106, including the end portion 120, side portion 130, motor 126, first capstan drum 122, second drum 144, torque cell 124, attachment member 152, controller 128, and multiple coupling mechanisms 148 and shafts 138. As shown and described the first capstan drum 122, second drum 144, and torque cell 124 may be aligned and supported between the end portion 120 and side portion 130, with the controller 128 mountable to the rear of the assembly.

Further, the lower portion 104 is depicted with the extension arms 112, each having a first end 142 and a second end 140, which are configured to attach to the cross brace 114. The cross brace 114 is shown with mount 150 for securing the pad 116, which provides ergonomic and cushioned support for the user's limb. The exploded view of FIG. 6 also shows the arrangement of coupling mechanisms 148 for securing the extension arms 112 to the cross brace 114, as well as for attaching the upper portion 102 components.

As further shown in FIG. 6, rotary encoder 146 may be disposed adjacent to the torque cell 124 and configured to provide displacement feedback corresponding to the output of the capstan drive. In some examples, rotary encoder 146 may be mounted coaxially with the first capstan drum 122 or torque cell 124 and may be operatively coupled to shaft 138. Additionally, motor 126 may comprise a rotary encoder positioned within the motor housing or as an external sensor coupled to the motor shaft. The encoder associated with motor 126 may be configured to provide high-resolution position feedback to the controller 128 and may support closed-loop control of torque and velocity. In some examples, the encoder may be used to provide proportional-integral-derivative (PID) input for field-oriented control (FOC) of motor 126. The arrangement of rotary encoder 146 and the encoder associated with motor 126 may support dual encoder feedback, wherein the encoder at the motor shaft provides a reference for commanded motion and the encoder at the capstan output captures the actual delivered motion to the patient interface. This configuration may be used to detect backlash, compliance, or hysteresis within the transmission system, and may further support diagnostic comparison between motor-side and output-side displacement.

As shown in FIG. 6, the proximal portion 142 of each extension arm 112 may include a flange 155 configured to couple to a corresponding surface 156 of the torque cell 124. An axle 153 may be disposed on the medial surface of the flange 155 and configured to engage a mounting hole 154 formed in the torque cell 124. The mounting hole 154 may be aligned with the output axis of the first capstan drum 122, which is operatively coupled to the torque cell 124. The axle 153 and mounting hole 154 may define a rotational axis about which the lower portion 104 of the brace pivots relative to the upper portion 102. In certain implementations, the mounting hole 154 may be positioned to correspond with the functional flexion-extension axis (FEA) of the user's knee when the brace is properly adjusted and fitted. The alignment of the axle 153 and mounting hole 154 with both the output axis of the capstan drum 122 and the anatomical FEA facilitates accurate torque transmission and measurement during knee articulation. Rotation of the capstan drum 122 may result in corresponding movement of the extension arms 112 about the defined axis, thereby enabling controlled flexion and extension of the user's leg.

The torque cell 124, capstan drum 122, and flange 155 of the extension arm 112 may be arranged in axial alignment to ensure consistent rotational behavior and torque measurement. The torque cell 124 may be configured to transmit torque generated by the capstan drive to the extension arms 112, which may articulate about the axis defined by the axle 153 and mounting hole 154. This configuration may allow the brace to replicate the natural biomechanics of the knee joint, particularly during flexion and extension movements. The spatial relationship between the flange 155, surface 156, and axle 153 may ensure that the extension arms 112 rotate about a fixed axis that corresponds to the user's physiological joint motion. In some examples, the flange 155 may be contoured, keyed, or otherwise shaped to mate with surface 156, thereby providing secure attachment and consistent rotational alignment. The arrangement of these components may reduce mechanical play and vibration, thereby increasing the accuracy and reliability of torque delivery and measurement.

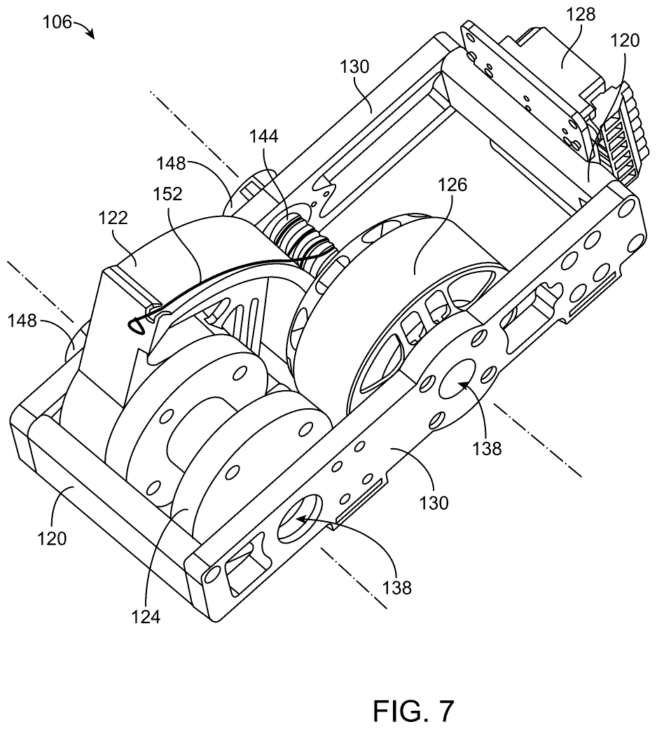

FIG. 7 illustrates an isometric view of the motor assembly 106 according to one aspect of the present disclosure. The motor assembly 106 is shown with its principal components assembled within the end portion 120 and side portion 130. As depicted, the assembly features two parallel axes. Located about the first axis is the first drum 122 and the torque cell 124. Located about the second parallel axis, is the second drum 144 and motor 126. The first drum 122 and torque cell 124 may be mounted to a first shaft 138, while the second drum 144 and motor 126 are mounted to a second shaft 138, where both shafts being anchored to side portion 130 via coupling mechanisms 148.

The arrangement of the first drum 122 and second drum 144 may form a winch or capstan drive, wherein the motor 126 directly drives the second drum 144. The attachment member 152, such as a flexible rope, cable, or belt, is wrapped multiple turns around the smaller-diameter second drum 144 and is fixed at each end to the larger-diameter first drum 122. This configuration mechanically couples the rotation of the two drums, such that actuation of the motor 126 and rotation of the second drum 144 results in corresponding movement of the first drum 122. The torque cell 124, mounted coaxially with the first drum 122, enables continuous measurement of the torque transmitted through the assembly.

In some aspects, the axes of the first and second drums may be parallel, and the attachment member 152 is routed in a manner that ensures the drums are “meshed” together, and in some cases operating analogously to a gear set. The ratio of the diameters of the first drum 122 to the second drum 144 determines the mechanical advantage, or effective gear reduction, of the assembly. This capstan drive arrangement provides several operational benefits, including smaller backlash, low inertia, high torque transparency, low noise, and cost-effective construction. Of course, in other examples, any suitable force transfer mechanism may be employed, such as gears, chain drives, etc.

In operation, rotation of the motor 126 may cause the second drum 144 to wind or unwind the attachment member 152, thereby rotating the first drum 122 and the attached torque cell 124. As the first drum 122 rotates, it in turn drives the first end 142 of the extension arm 112, enabling controlled movement of the LIMB 100 in response to motor commands.

In some embodiments, the capstan drum 122 may include a drum surface 160 defined along its outer rim, extending between an uppermost point 161 and a lowermost point 162. The drum surface 160 may span a circumferential angle that corresponds to a portion of a full circle, such as approximately ninety percent in the illustrated example. The angular span between points 161 and 162 may define the range of motion permitted by the brace, with point 161 positioned closest to the motor 126 axis when the patient's leg is fully extended and point 162 positioned closest to the motor 126 axis when the leg is bent. In various implementations, the angular span of drum surface 160 may be reduced to accommodate limited mobility, such as thirty percent, fifteen percent, or even as little as five or six percent for patients with severe knee injuries or post-operative restrictions. The positions of 161 and 162 may be used to define both the starting and ending angles of knee articulation, allowing the brace to be configured for specific rehabilitation protocols or diagnostic assessments. In some examples, the attachment member 152 may be adjusted in length to further constrain or expand the allowable bend range. The system may include a user interface or clinician-accessible control module enabling configuration of these parameters. For instance, controller 128 may include executable code that allows the user or clinician to define rotational parameters of motor 126, which in turn govern the rotational behavior of drum 122 and the corresponding torque delivery profile. This configurability supports tailored treatment plans and targeted biomechanical testing across a range of clinical scenarios.

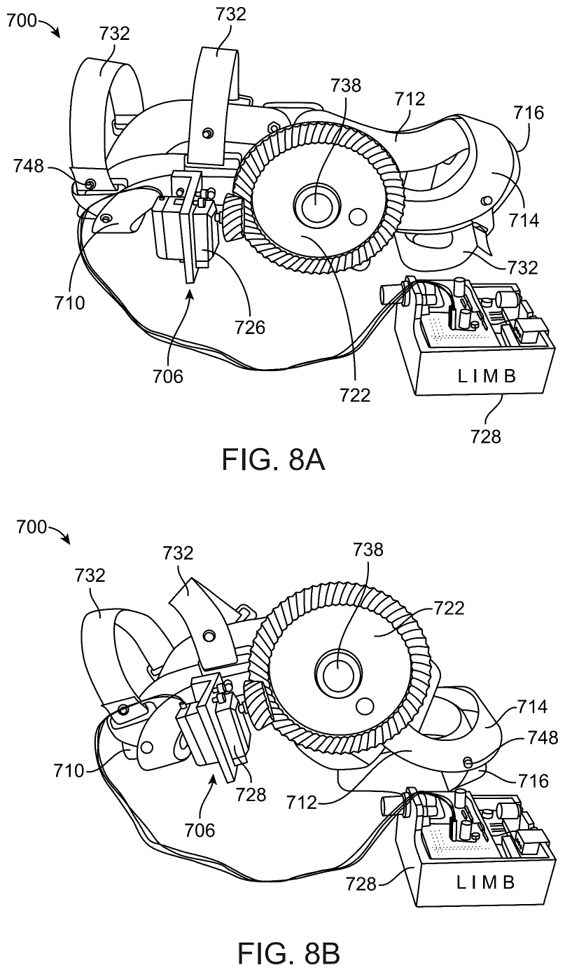

FIGS. 8A and 8B illustrate another LIMB 700 as described herein. The LIMB 700 as shown in FIGS. 8A and 8B includes a motor assembly 706 having a drive motor 726, a drum 722, and a shaft 738, a rear bracket 710, one or more extension arms 712, a cross brace 714, pads 716, one or more straps 732 coupled throughout the LIMB 700, a controller 728 and a plurality of coupling mechanisms 748.

FIGS. 8A and 8B illustrate another embodiment of a Leg Isoinertial Measurement Brace, LIMB 700, in accordance with the present disclosure. LIMB 700 includes a motor assembly 706 comprising a drive motor 726, a drum 722, and a shaft 738. The drive motor 726 is operatively coupled to the drum 722 through the shaft 738, enabling controlled rotation of the drum 722 under the direction of a controller 728. The motor assembly 706 may be secured in place using a plurality of coupling mechanisms 748.

The LIMB 700 further includes one or more extension arms 712 extending from the motor assembly 706 or rear bracket 710 to a cross brace 714 positioned at the lower portion of the device. The cross brace 714 is provided with one or more pads 716 to enhance user comfort and distribute pressure, while one or more straps 732 may be routed throughout the LIMB 700 to secure the device to the user's leg. The straps 732 may be adjustable and removable, facilitating a customizable and secure fit. Additionally, the controller 728 may be mounted near upper portion of LIMB 700 and may be configured to regulate the operation of the drive motor 726 and process sensor inputs, if present.

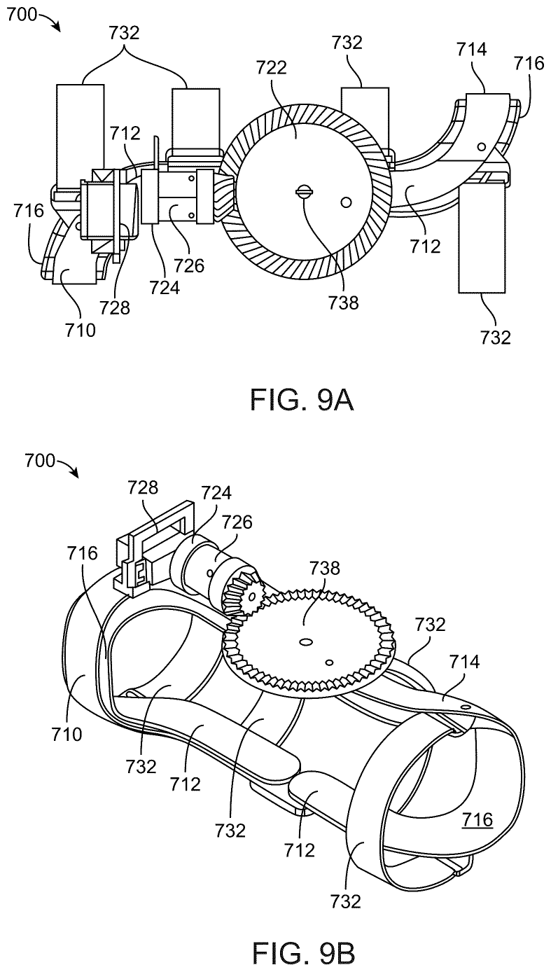

FIGS. 9A and 9B illustrate a similar LIMB 700 as described with respect to FIGS. 8A and 8B. Here the LIMB 700 is shown from a side view in FIG. 9A and a isometric angled view in 9B. s with the previous embodiment, LIMB 700 includes a motor assembly 706 comprising a drive motor 726, a drum 722, and a shaft 738, along with associated structural components such as the rear bracket 710, extension arms 712, cross brace 714, pads 716, straps 732, controller 728, and coupling mechanisms 748. In the example of FIGS. 9A and 9B, the motor assembly 706 further includes a gear train operatively coupled between the drive motor 726, the drum 722, and the shaft 738. The gear train is configured to transmit rotational motion from the drive motor 726 to the drum 722, thereby enabling the controlled rotation of the drum 722. In other examples, this configuration may allow the controller 728 to accurately regulate limb movement and resistance by controlling the drive motor 726 and the associated gear train, thus meeting the functional requirements for rehabilitation, measurement, or assistive applications as intended by the present disclosure. It should be noted that the LIMB 700 as described with respect to FIGS. 8A, 8B, 9A, and 9B may incorporate other elements of the LIMB 100 or other LIMB examples herein.

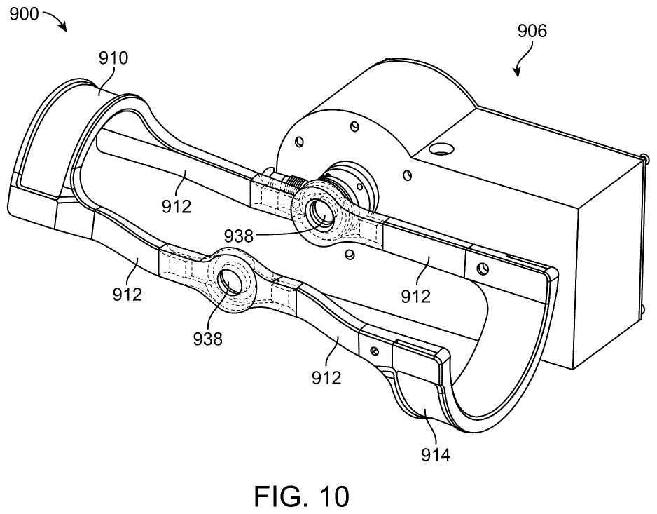

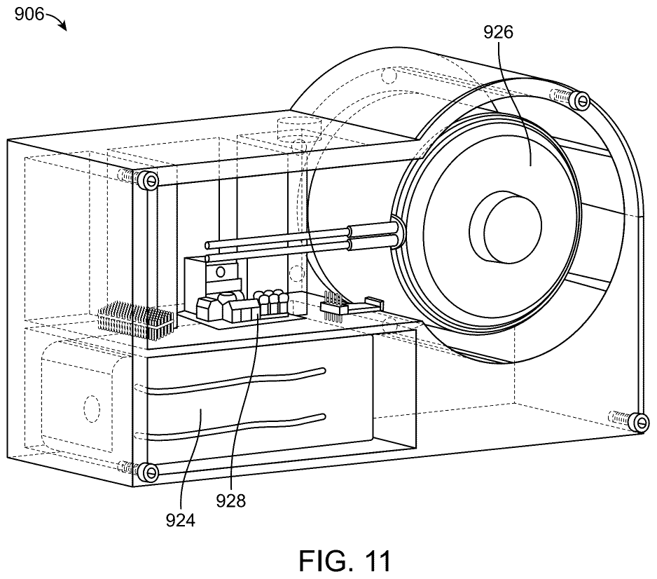

FIGS. 10 and 11 illustrate another example LIMB 900 according to aspects of the present disclosure. Here, the LIMB 900 may include various aspects of the other LIMB examples as described herein. FIG. 10 includes the LIMB 900 with a motor assembly 906, one or more extension arms 912, a rear bracket 910, a cross brace 914. Here the one or more extension arms 912 may be coupled to one another with one or more shafts 938.

FIG. 11 specifically illustrates the motor assembly 906 of the LIMB 900 of FIG. 10. Similar to the other LIMBs described herein, the motor assembly 906 may include a motor 926 located within a protective housing, a torque cell 924, and a controller 928. Various aspects of the motor assembly 906 may be housed or coupled to the LIMB 900 using attachment means described herein.

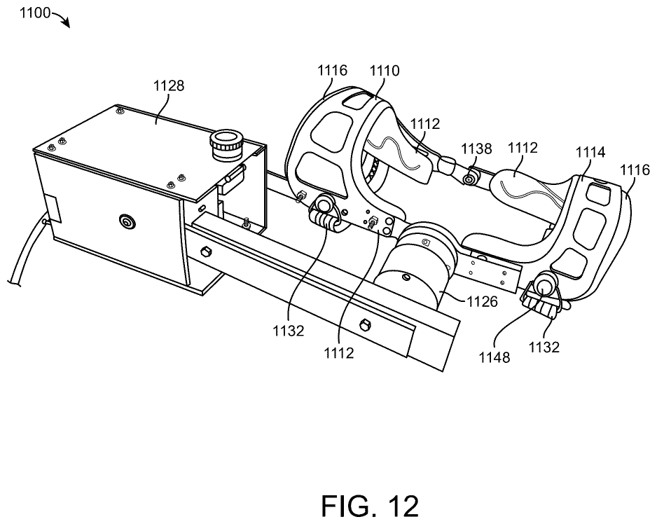

FIG. 12 illustrates an additional LIMB 1100, similar to other examples as described herein. The LIMB 1100 may include a cross brace 1114 with a pad 1116, a rear bracket with a pad 1116, one or more extension arms 1112 rotatably coupled about a shaft 1138, one or more straps 1132, a motor 1126, and a controller 1128.

As shown, LIMB 1100 includes a cross brace 1114 positioned at one end of the device, with a pad 1116 attached to provide a cushioned contact surface for the user's limb. A rear bracket 1110 is also included, which may be provided with an additional pad 1116 to further enhance user comfort and distribute pressure. The cross brace 1114 and rear bracket 1110 are spaced apart and connected by one or more extension arms 1112. The extension arms 1112 may be rotatably coupled about a shaft 1138, allowing angular adjustment or articulation. The shaft 1138 may be supported by bearings or bushings to enable smooth and controlled movement.

The LIMB 1100 further comprising a motor 1126 may be operatively coupled to the extension arms 1112, enabling powered movement or resistance during use. The motor 1126 may be arranged to drive the shaft 1138 directly or via a gear or linkage mechanism, providing controlled motion or resistance in response to user input or pre-programmed protocols. A controller 1128 may be provided, typically housed in a protective enclosure, and is configured to regulate the operation of the motor 1126, process sensor inputs, and execute control algorithms tailored for isoinertial measurement and rehabilitation applications. In some examples, the controller 1128 may include user interface elements, such as adjustment knobs or buttons, and may support communication with external devices for data transfer, monitoring, or configuration.

As shown, one or more straps 1132 are provided to secure the LIMB 1100 to the user's limb. The straps 1132 may be routed through or around the cross brace 1114, rear bracket 1110, or extension arms 1112, The LIMB 1100 may also include a plurality of coupling mechanisms 1148, such as bolts, screws, or quick-release fasteners, to facilitate assembly, adjustment, and maintenance of the device.

FIG. 13 illustrates an example control system 1200 that may be used in conjunction with any of the LIMB devices described herein, such as the LIMB 100, LIMB 700, LIMB 900 or LIMB 1100. In some aspects, the control system 1200 may be configured to provide closed-loop control, data acquisition, and user interface functionality for the LIMB assemblies. The control system 1200 may include a laptop 1202, microcontroller and/or Bluetooth module 1218, a motor 1216, a torque cell 1212, and one or more rotary encoders (1214, 1222) for displacement and position feedback. The rotary encoders 1214 and 1222 may be configured to operate at different rotational ratios, such that encoder 1214 may be coupled to the motor 1216 for PID motor control, while encoder 1222 may be coupled to the output shaft or extension arm for displacement measurement. In some examples, the microcontroller 1218 may compare the outputs of encoders 1214 and 1222 to detect mechanical backlash, wherein a discrepancy in movement between the two encoders may indicate slack or play in the transmission system. Upon detecting such backlash, the microcontroller 1218 may trigger a tensioning adjustment protocol to restore mechanical integrity and ensure accurate torque transmission.

Within some examples, the control system 1200 may include a battery 1204 as a portable power source, which may be regulated by a DC-DC step-down converter logic circuit 1206 to provide appropriate voltage and current levels for the system components. The control system 1200 may further include an amplifier or signal conditioner 1210, which may be used to process analog signals from the torque cell 1212 prior to digitization. The processed analog signals may then be converted to digital format by an analog-to-digital converter (ADC) 1280, enabling precise measurement and monitoring of force or torque applied during device operation. In some examples, the control system 1200 may implement closed-loop field-oriented control (FOC) with a proportional-integral-derivative (PID) algorithm executed by the microcontroller 1218. The FOC-PID control may utilize feedback from the torque cell 1212 and rotary encoders 1214, 1222 to regulate motor 1216 operation with high precision, minimizing jitter and vibration during actuation.

In some aspects, the motor 1216 may be controlled by the microcontroller 1218 based on feedback from the torque cell 1212 and rotary encoders 1214, 1222. The rotary encoders may provide high-resolution displacement output and PID input for accurate position and velocity control of the motor 1216. The microcontroller 1218 may execute control algorithms, such as PID control, to achieve desired movement profiles or resistance levels. The control system 1200 may also include an emergency switch 1220 for user safety, which may immediately disable the motor 1216 or place the system in a safe state when activated. In some examples, the encoder feedback from 1214 and 1222 may be used not only for motor control but also for real-time monitoring of system performance, including torque delivery accuracy and mechanical compliance.

Still referring to FIG. 13, the control system 1200 may support wireless or wired communication with an external computing device, such as a laptop 1202. The laptop 1202 may be configured to receive input and output signals via the microcontroller and/or Bluetooth module 1218, enabling bidirectional data exchange. In some examples, the laptop 1202 may be used to configure system parameters, upload or modify torque profiles, monitor real-time data streams including encoder readings and torque measurements, log performance metrics, or update firmware. The output from the control system 1200 to the laptop 1202 may include real-time graphical representations of torque versus time, encoder displacement, motor status, and system alerts. The input from the laptop 1202 may include user-defined presets, safety thresholds, or calibration routines. The system may also be capable of remote monitoring or control through the Bluetooth module or other wireless interfaces. The modular and flexible nature of the control system 1200 allows it to be adapted for a variety of LIMB configurations and user requirements, supporting a wide range of rehabilitation, measurement, and assistive applications. Alternative implementations may include integration with cloud-based platforms, mobile devices, third-party rehabilitation software, etc.

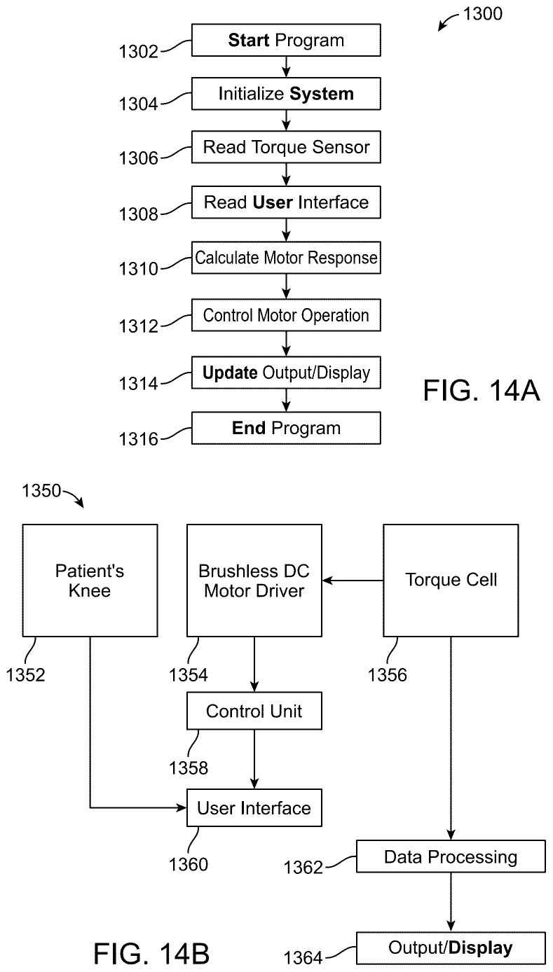

FIG. 14A illustrates an example method for the control system of a LIMB, such as the control systems or controllers as described herein. At 1302, the program may begin on a LIMB device or controller.

At 1304, the system may be initialized. In some examples, initialization may include powering on the control electronics, performing self-checks of sensors and actuators, establishing communication with a user interface or external computing device, and loading any preconfigured parameters or control algorithms.

At 1306, the system may receive or read a torque sensor. This may involve acquiring analog or digital signals from a torque cell or force sensor, optionally conditioning the signal via an amplifier or signal conditioner, and converting the measurement to a digital value using an analog-to-digital converter (ADC). In some aspects, the system may filter or calibrate the sensor data to improve accuracy and reliability.

At 1308, the system may read a user interface. In some examples, the user interface may include physical buttons, dials, a touchscreen, or a remote computing device such as a laptop or mobile phone. The system may receive user input specifying desired resistance, movement profiles, therapy modes, or safety settings.

At 1310, the system may calculate motor response. This may include executing control algorithms, such as proportional-integral-derivative (PID) control, to determine motor commands based on the measured torque, user input, and any pre-set limits or profiles. The calculation may also incorporate feedback from position encoders or other sensors. In some examples, the motor response may be adjusted to match a predefined torque-time profile, such as a linear or exponential ramp, or to maintain a target torque level during a static hold. Adjustments may also be made to facilitate the generation of stiffness curves, to detect deviations indicative of joint instability, or to support the execution of rehabilitation protocols that require progressive loading. In certain implementations, the motor response may be modified to maintain consistent torque application across repeated trials or to accommodate subject-specific parameters.

At 1312, the system may control motor operation. In some aspects, this may involve sending commands to the motor driver circuitry to adjust speed, direction, or torque output. The system may also monitor for fault conditions, such as overcurrent, overheating, or emergency stop activation, and respond accordingly.

At 1314, the system may update the output or the display. This may include providing real-time feedback to the user via a graphical or textual display, updating performance metrics, and logging data for later analysis. In some examples, the system may transmit data to an external computing device or cloud service for monitoring, storage, or remote supervision. The method ends at 1316.

FIG. 14B illustrates another example system 1350 for use with a patient, such as for isoinertial measurement or rehabilitation applications. In some aspects, system 1350 may be integrated with any of the LIMB devices described herein. As shown, the system 1350 includes an interface to a patient's knee 1352, which may be mechanically coupled to a brushless DC motor driver 1354. The motor driver 1354 may be configured to provide controlled actuation of the LIMB device in response to control signals.

In some examples, a torque cell 1356 is operatively connected to the brushless DC motor driver 1354 and is configured to sense the torque or resistance encountered during patient movement or device operation. The output from the torque cell 1356 may be transmitted to a data processing module 1362, which may filter, analyze, and store the measured data for further evaluation. The torque data may also be used in real-time by the control unit 1358 to adjust the operation of the motor driver 1354, providing closed-loop feedback and enabling precise control of the therapeutic or measurement protocol.

The system 1350 further includes a control unit 1358, which may be implemented as a microcontroller, processor, or embedded system. Various aspects of other controllers as described herein may be implemented. The control unit 1358 may receive inputs from the patient's knee 1352, the brushless DC motor driver 1354, and the torque cell 1356, and may execute control algorithms to regulate device operation. A user interface 1360 may be provided to enable user or clinician interaction with the system, allowing for configuration of parameters, initiation of therapy sessions, or monitoring of patient progress. The processed data may be output to an output or display device 1364, which may present relevant information such as torque profiles, session summaries, or real-time feedback to the user or clinician.

In some examples, the system 1350 may be configured for wireless or wired communication with external devices, enabling remote monitoring, data logging, or integration with electronic medical records. The modular design of the system 1350 allows it to be adapted for various clinical, therapeutic, or research applications, supporting a wide range of user needs and operational scenarios.

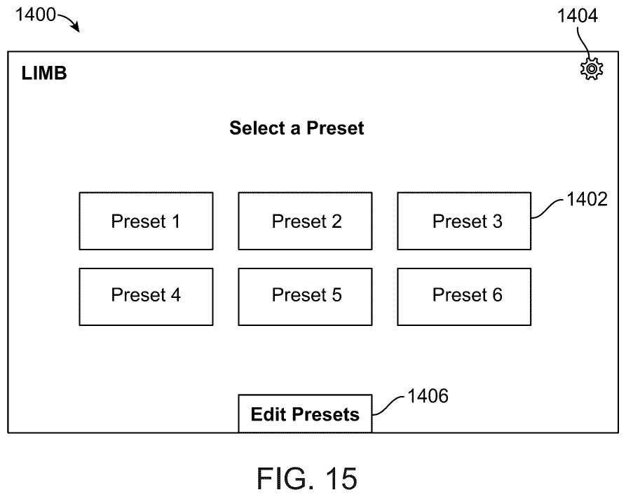

FIGS. 15-19 illustrate the preset and testing display 1400 for a LIMB using one or more of the systems as described herein. The process display 1400 may be used alongside any of the LIMB devices as described herein as well as one or more of the control system or methods.

FIG. 15 illustrates an example preset display 1400 which may be located on a user interface. Here, the preset display 1400 may include setting options 1404, one or more presets 1402, and an option to edit presets 1406. As shown, a user may be prompted to select a preset and design a test for a user. In some examples, the display 1400 may be implemented on a touchscreen device, tablet, or computer, and may support remote or wireless communication with the LIMB device. The graphical and interactive nature of the display 1400 enables efficient and user-friendly creation of complex test protocols, supporting a wide range of clinical, research, or training applications.

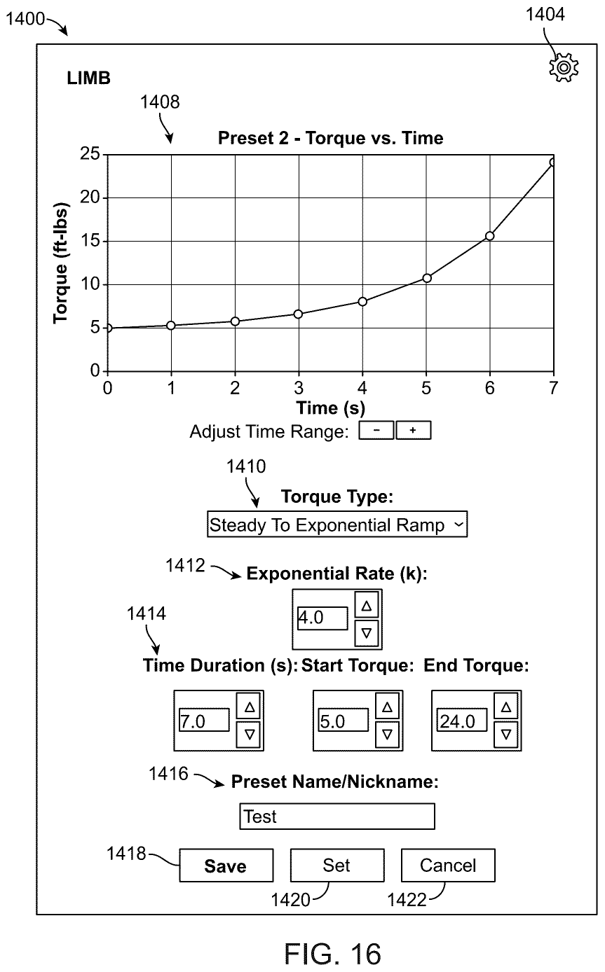

FIG. 16 illustrates an example display 1400 capable of creating a preset for a LIMB test. Like the display shown in FIG. 15, the preset display 1400 may include an options or settings icon 1404, which may be selected to access additional configuration parameters or system preferences. The display 1400 is configured to provide an intuitive graphical user interface for creating, modifying, and saving test presets for use with any of the LIMB devices described herein.

As shown, the preset display 1400 may present a graph 1408, which visually represents a torque versus time profile corresponding to the current preset configuration. The graph 1408 allows the user to preview the expected output of the LIMB device during the test, providing immediate visual feedback as parameters are adjusted. In some examples, the user may interactively adjust the time range or other graph axes to tailor the display to their preferences or the requirements of a specific test protocol by editing the preset.

The preset may be defined using one or more selectable parameters, such as a torque type 1410, exponential rate 1412, and time 1414. The torque type 1410 may include options such as steady, ramp, exponential ramp, or other predefined or custom torque profiles. The exponential rate 1412 enables the user to specify the rate constant for an exponential torque profile, allowing fine-tuned control over the shape and progression of the torque curve. The time 1414 parameter may include start and end times, as well as start and end torque values, permitting the user to define the duration and magnitude of the test or exercise.

Once the desired preset parameters have been configured, the user may assign a preset name or nickname 1416 for easy identification and future retrieval. At the bottom of the preset display 1400, the user interface presents options to save 1418, set 1420, or cancel 1422 the current preset or graph settings. The save option 1418 may store the preset in memory for later use, the set option 1420 may immediately apply the preset to the LIMB device for the upcoming test or session, and the cancel option 1422 may discard changes or exit the preset creation interface. In some aspects, the display 1400 may also provide additional prompts or confirmations to ensure that presets are properly configured and stored.

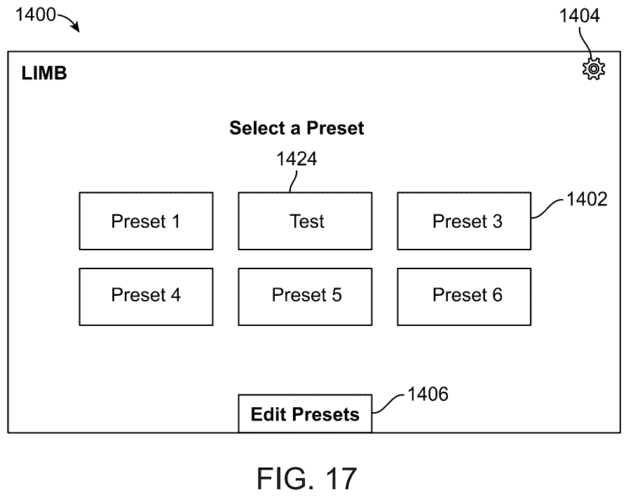

FIG. 17 illustrates the display 1400 with a main interface for selecting a test or preset, such as test 1424, which may correspond to a previously configured set of parameters as described with respect to FIG. 16. The display 1400 may present a series of selectable presets 1402, allowing the user to quickly choose from stored protocols for different testing or rehabilitation scenarios. In some aspects, the interface includes an edit presets option 1406, enabling the user to modify an existing preset, such as test 1424, by adjusting parameters or updating the configuration based on new clinical requirements or user preferences.

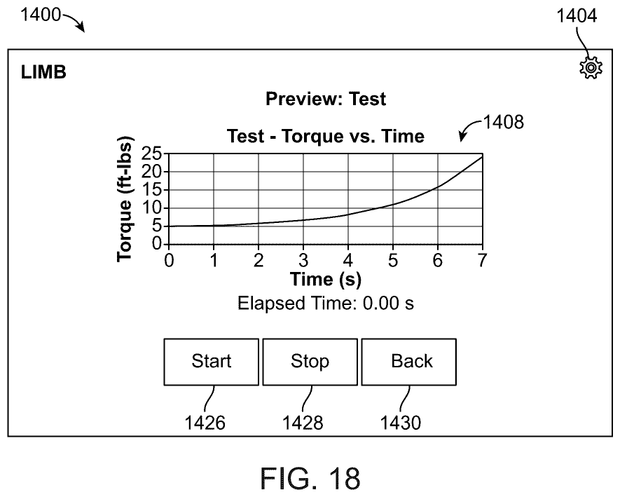

FIG. 18 illustrates a test preview on a display 1400. Here the preview may be a graph 1408 showing the parameters selected with respect to FIG. 16. In some cases, one or more of the selectable parameters, such as a torque type 1410, exponential rate 1412, and time 1414 may also be displayed on the display 1400 test preview. As shown within FIG. 18, a user may be able to start 1426, stop 1428, or go back 1430 to selected presets before running a test on a user using a LIMB device as described herein.

FIG. 18 illustrates a test preview screen on display 1400, providing the user with a graphical representation 1408 of the selected preset's torque versus time profile prior to initiating the test. The preview may display the key parameters, such as torque type 1410, exponential rate 1412, and time 1414, allowing for final review and confirmation before proceeding. In some cases this preview function may help ensure that the user or clinician can verify the intended protocol and make any necessary adjustments for a specific user. In some examples, the test preview display 1400 may provide interactive controls, including start 1426, stop 1428, and back 1430 buttons, enabling the user to begin the test, halt it if needed, or return to the preset selection screen. training environments.

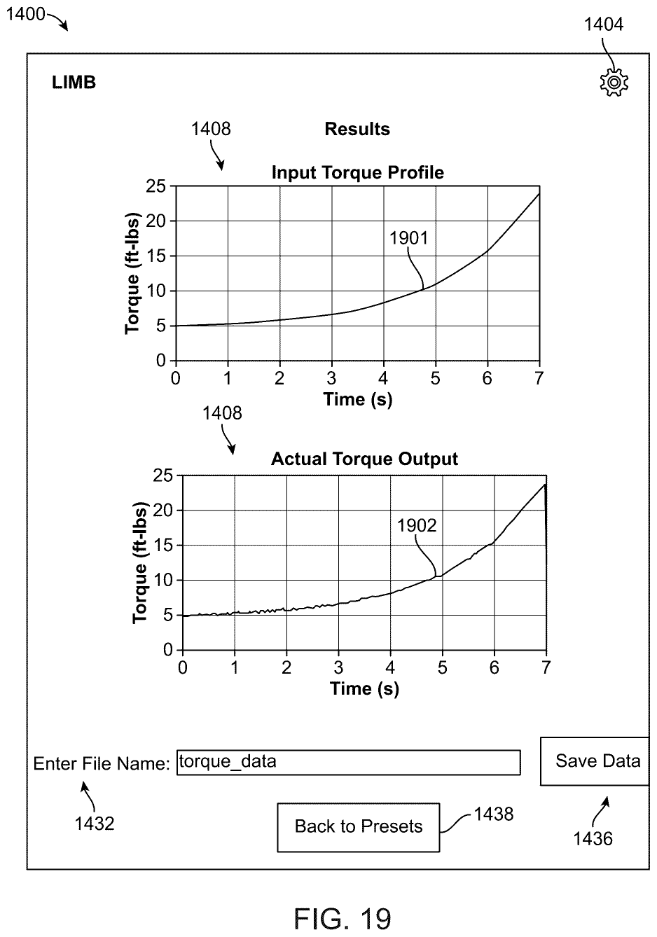

FIG. 19 is a display 1400 configured to compare one or more graphs, such as the input torque profile preset and the actual torque output measured during a test run on a user. The display 1400 may be presented on a touchscreen device, computer, or dedicated user interface, and may include an options or settings icon 1404 for accessing additional display or data management features.

As shown, the display 1400 may include a first graph 1408, labeled “Input Torque Profile,” which represents the preset torque versus time curve that was programmed into the LIMB device prior to the test. Directly below, a second graph 1408 is provided, labeled “Actual Torque Output,” which displays the measured torque output generated by the LIMB device during the test session. The side-by-side or stacked presentation of these graphs allows a user, clinician, or researcher to visually compare the intended test protocol with the real-world performance, facilitating rapid assessment of device accuracy, user compliance, or system tuning.

In some examples, the display 1400 may further prompt the user to enter a file name 1432 in a designated field, enabling easy organization and retrieval of test results. Once a suitable file name has been entered, the user may select a “Save Data” button 1436 to store the recorded torque data, graphs, and relevant metadata to local or networked storage. After saving, the interface may prompt the user to return to the preset selection screen via a “Back to Presets” button 1438, allowing for efficient workflow if additional tests are to be conducted or new protocols are to be configured. In some aspects, the display 1400 may also provide additional feedback, such as confirmation messages, error notifications, or summary statistics, to further enhance the user experience and ensure data integrity.

In this particular example, FIG. 19 illustrates a dual-graph display interface configured to compare a preset input torque profile 1901 with the actual torque output profile 1902 measured during a test session. The upper graph, corresponding to torque input profile 1901, represents the programmed torque versus time curve, which in this example follows an exponential ramp beginning at 5 foot-pounds and increasing with a rate of change greater than one. The lower graph, corresponding to torque output profile 1902, displays the real-time torque response generated by the LIMB device. During the first two seconds of the test, the wearer exhibited jitter —characterized by rapid, involuntary fluctuations in torque output—while attempting to stabilize the limb under low torque conditions. By the fourth second, the wearer was able to maintain a steady angular knee position as torque increased from 8 to 23 foot-pounds, demonstrating improved neuromuscular control under higher load.

The LIMB system may be configured to instruct the user to maintain a specific joint angle or range of motion during the application of torque. In some examples, the user may be directed to hold a slight bend in the knee or another predetermined angle. Deviations from the instructed angle may result in observable fluctuations in the torque output profile, which may be used to assess the user's ability to maintain joint stability under load. These deviations may be interpreted by a clinician as indicators of fatigue, instability, or recovery status. The system may further support the creation of customized torque profiles, including ramp, exponential, or oscillatory patterns, enabling tailored assessment protocols for individual users. The ability to compare input and output torque profiles in real time provides a robust framework for evaluating joint mechanics and neuromuscular performance.

The LIMB system may be deployed across a variety of clinical and operational environments, including rehabilitation clinics, sports medicine facilities, military installations, and eldercare institutions. In one example, a clinician may establish a baseline torque response profile for a user during an initial evaluation. Following an injury or surgical intervention, the same user may be retested using identical or modified torque profiles, allowing for direct comparison to the baseline and quantification of recovery progress. In military applications, the system may be used to assess joint readiness and injury severity in soldiers. In nursing homes, the device may assist in monitoring joint stability and fall risk among elderly residents. Sports teams may utilize the system to evaluate performance, track rehabilitation, and optimize training protocols for athletes. The system's modular design and programmable control algorithms support a wide range of use cases and user populations.

In some examples, the LIMB system may be configured to generate both positive and negative torque profiles. Positive torque may correspond to limb extension or straightening, while negative torque may simulate resistance against flexion or curling. This bidirectional capability enables comprehensive testing of joint function and muscle coordination. For instance, a rehabilitation protocol may include alternating torque directions to evaluate concentric and eccentric muscle control. The system may also support angle-dependent torque profiles, wherein the applied torque varies as a function of joint angle. This feature allows physical therapists to design protocols that target specific muscle groups and movement patterns, replacing traditional resistance bands with programmable, motor-driven resistance that can be finely tuned to the desired therapeutic outcome.

The LIMB system may further include sensors and control algorithms configured to measure and regulate the angular position of the limb during operation. In some examples, the user may be instructed to perform specific movements, such as walking with the brace, straightening the leg, or curling the leg, while the system monitors and records the corresponding torque and angle data. The system may be programmed to apply torque in a manner that is dependent on the measured joint angle, enabling precise control over resistance at specific points in the range of motion. This capability allows clinicians to prescribe targeted exercises that isolate particular muscle groups or joint behaviors. For example, a physical rehabilitation practitioner may define a torque profile that increases resistance as the knee approaches full extension, thereby strengthening the quadriceps. Alternatively, resistance may be applied during flexion to engage the hamstrings. The programmable nature of the system provides enhanced control and repeatability compared to conventional elastic bands or manual resistance techniques.

As used herein, anatomical directional terms such as “proximal,” “distal,” “medial,” “lateral,” “anterior,” “posterior,” and the like are intended to have their conventional meanings as understood in the medical and anatomical arts, unless the context clearly dictates otherwise. These terms are used to describe the relative positions and orientations of components with respect to a user's body when the device is worn or applied. For example, “proximal” may refer to a location closer to the torso, while “distal” may refer to a location farther from the torso. Similarly, “medial” and “lateral” may refer to positions toward or away from the midline of the body, respectively. These terms are not intended to be limiting and may be interpreted in accordance with standard anatomical conventions applicable to human or animal subjects. Where appropriate, directional terminology may also be used to describe the spatial relationships between device components or features. All such terminology is to be construed in a manner consistent with the intended function and orientation of the device during use.

It is to be understood that the disclosure is not limited in its application to the details of construction and the arrangement of components set forth in the following description or illustrated in the following drawings. The disclosure is capable of other examples and of being practiced or of being carried out in various ways. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

It should be noted that the methods described herein describe possible implementations. The operations and the steps may be rearranged, or otherwise modified and other implementations are possible. Further, aspects from two or more of the methods may be combined.

The various illustrative blocks and components described in connection with the disclosure herein may be implemented or performed using a general-purpose processor, a DSP, an ASIC, a Central Processing Unit (CPU), a graphics processing unit (GPU), a neural processing unit (NPU), a Field Programmable Gate Array (FPGA) or other PLD, a quantum processor, a discrete gate or transistor logic, discrete hardware components, or any combination thereof designed to perform the functions described herein. A general-purpose processor may be a microprocessor but, in the alternative, the processor may be any processor, controller, microcontroller, or state machine. A processor may also be implemented as a combination of computing devices (for example, a combination of a DSP and a microprocessor, multiple microprocessors, one or more microprocessors in conjunction with a DSP core, a combination of classical and quantum processors, or any other such configuration). Any functions or operations described herein as being capable of being performed by a processor may be performed by multiple processors that, individually or collectively, are capable of performing the described functions or operations.

The functions described herein may be implemented using hardware, software executed by one or more processors, firmware, or any combination thereof. If implemented using software executed by multiple processors, the functions may be stored as or transmitted using one or more instructions or code of a computer-readable medium. Other examples and implementations are within the scope of the disclosure and appended claims. For example, due to the nature of software, functions described herein may be implemented using software executed by one or more processors, hardware, controllers, firmware, hardwiring, circuitry, or combinations of any of these. Features implementing functions may also be physically located at various positions, including being distributed such that portions of functions are implemented at different physical locations.

Computer-readable media includes both non-transitory computer storage media and communication media including any medium that facilitates transfer of a computer program from one location to another. A non-transitory storage medium may be any available medium that may be accessed by a general-purpose or special-purpose computer, whether classical or quantum. By way of example, and not limitation, non-transitory computer-readable media may include random access memory (RAM), read-only memory (ROM), electrically erasable programmable ROM (EEPROM), flash memory, compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer or a general-purpose or special-purpose processor. Also, any connection may be properly termed a computer-readable medium. For example, the software may be transmitted from a website, server, or other remote source using a wired technology such as a coaxial cable, fiber optic cable, twisted pair, digital subscriber line (DSL), universal serial bus (USB), high-definition multimedia interface (HDMI), video graphics array (VGA), digital visual interface (DVI), thunderbolt cable, power cable, ribbon cable, integrated services digital network (ISDN), or wireless technologies such as wireless fidelity (Wi-Fi), Bluetooth, cellular network, near-field communication (NFC), Zigbee, long range (LoRa), infrared (IR), radio frequency identification (RFID), light fidelity (Li-Fi), satellite, ultra-wideband (UWB), millimeter wave (mmWave), and microwave. The wired and or wireless technologies are included in the definition of computer-readable medium. Disk and disc, as used herein, include a compact disk (CD), laser disc, optical disc, digital versatile disc (DVD), floppy disk, and Blu-ray disc. Disks or discs may reproduce data magnetically or optically using lasers. Combinations of the above are also included within the scope of computer-readable media. Any functions or operations described herein as being capable of being performed by a memory may be performed by multiple memories that, individually or collectively, are capable of performing the described functions or operations.

As used herein, the term “computing” may refer to any operations that may be performed by a computer (or a computing device), including (but not limited to): computation, data storage, data retrieval, communication, execution of an algorithm, and the like. Further, as used herein, a “computing device” may refer to any device in which a computing operation may be carried out. A computing device may be, for example (but not limited to): a compute component, a storage component, a network device, a telecommunications component, and the like.

As used herein, the term “computing resource” may refer to any program, application, document, asset, executable program file, desktop environment, computing environment, network environment, or other resource made available to, for example, a user of a computing device. A computing resource may be delivered to a computing device via, for example (but not limited to): conventional installation, a method of streaming, a virtual machine executing on a remote computing device, execution from a removable storage device connected to the computing device (e.g., a universal serial bus (USB) device), and the like.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof. As used herein, outputting at least one signal may refer to any type of signal that may be output, including wireless communication signals, electrical signals, or any other type of signal that may be transferred by voltages, currents, electromagnetic waves, magnetic fields or particles, optical fields or particles, or any combination thereof. As used herein, “communication” may refer to data transferring or passing, or may refer to two or more components coordinating a job or task. As used herein, the term “data” is intended to be broad in scope. In this manner, that term “data” embraces, for example, (but not limited to): a data stream (or stream data), data chunks, data blocks, atomic data, objects of any type, files of any type (e.g., media files, spreadsheet files, database files, etc.), directories, sub-directories, volumes, and the like.

Machine learning includes one or more systems that may be adapted to generate, train, and execute one or more trained learning models, nodes, neural networks, gradient boosting algorithms, mutual information classifiers, random forest classifications, and other machine learning and artificial intelligence-based algorithms or models to process inputs, parameters, and other data elements. In some examples, the one or more trained learning models can include deep learning, machine learning, neural networks, computer vision, and similar advanced artificial intelligence-based technologies. In some examples, the machine learning or artificial intelligence may use additional inputs and/or feedback loops to an iterative training process for enhanced data processing and improved outcomes.

Although the disclosure may describe components and functions that may be implemented in a particular example with reference to a particular standard or protocol, the disclosure is not limited to the standard or protocol. Other standards or protocols supporting similar functionality are considered equivalents thereof.

As used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Furthermore, “and/or” as used in a list of items indicates an inclusive list such that, for example, a list of at least one of A, B, and/or C means A or B or C or AB or AC or BC or ABC (i.e., A and B and C). Also, as used herein, the phrase “based on” shall not be construed as a reference to a closed set of conditions. For example, an example step that may be described as “based on condition A” may be based on both a condition A and a condition B. For example, as used herein, the phrase “based on” shall be construed in the same manner as the phrase “based at least in part on.”

As used herein, including in the claims, the article “a” before a noun may be open-ended and understood to refer to “at least one” of those nouns or “one or more” of those nouns. Thus, the terms “a,” “at least one,” “one or more,” and “at least one of one or more” may be interchangeable. For example, if a claim recites “a component” that performs one or more functions, each of the individual functions may be performed by a single component or by any combination of multiple components. Thus, the term “a component” having characteristics or performing functions may refer to “at least one of one or more components” having a particular characteristic or performing a particular function. Subsequent reference to a component introduced with the article “a” using the terms “the” or “said” may refer to any or all of the one or more components. For example, a component introduced with the article “a” may be understood to mean “one or more components,” and referring to “the component” subsequently in the claims may be understood to be equivalent to referring to “at least one of the one or more components.” Similarly, subsequent reference to a component introduced as “one or more components” using the terms “the” may refer to any or all of the one or more components. For example, referring to “the one or more components” subsequently in the claims may be understood to be equivalent to referring to “at least one of the one or more components.”

The terms “determine,” “determining,” “identify,” or “identifying” encompasses a variety of actions and, therefore, “determining” or “identifying” can include calculating, computing, processing, deriving, investigating, looking up (such as via looking up in a table, a database, or another data structure), receiving, ascertaining, and the like. Also, “determining” or “identifying” can include receiving (for example, receiving information), accessing (for example, accessing data stored in memory), retrieving, and the like. Also, “determining” or “identifying” can include resolving, obtaining, selecting, choosing, establishing, and other such similar actions.

In the appended figures, similar components or features may have the same reference label. Further, various components of the same type may be distinguished by following the reference label by a dash and a second label that distinguishes among the similar components. If just the first reference label may be used in the specification, the description may be applicable to any one of the similar components having the same first reference label irrespective of the second reference label or other subsequent reference label.

The description set forth herein, in connection with the appended figures, describes example configurations and does not represent all the examples that may be implemented or that are within the scope of the claims. The term “example” or “instance” used herein means “serving as an example, instance, or illustration” and not “preferred” or “advantageous over other examples.” The detailed description includes specific details for the purpose of providing an understanding of the described techniques. These techniques, however, may be practiced without these specific details. In some figures, known structures and devices are shown in block diagram form in order to avoid obscuring the concepts of the described examples. The features of the various examples described herein may be combined in any suitable manner. It is contemplated that one or more features from one example may be incorporated into another example unless explicitly stated otherwise. The combinations of features from different examples are within the scope of the disclosure.

Although terms such as “document,” “file,” “segment,” “block,” or “object” may be used by way of example, the present disclosure is not limited to any particular form of representing and storing data or other information. Rather, the present disclosure may be equally applicable to any object capable of representing information.

It will be appreciated by those skilled in the art that while the disclosure has been described above in connection with particular examples, the disclosure is not necessarily so limited, and that numerous other examples, uses, means for, modifications and departures from the examples, uses, and means for are intended to be encompassed by the claims attached hereto. The entire disclosure of each patent and publication cited herein is incorporated by reference, as if each such patent or publication were individually incorporated by reference herein. Various features and advantages of the disclosure are set forth in the following claims.

What is claimed is:

1. A device, comprising:
an upper member adapted to fit a wearer's upper leg;
a lower member adapted to fit a wearer's lower leg;
wherein the upper member and the lower member are rotationally coupled about a rotational axis;
a torque cell operatively coupled to the upper member or the lower member and configured to measure torque at the rotational axis;
a drive assembly configured to transmit torque to the upper member and the lower member about the rotational axis;
a motor assembly coupled the drive assembly to actuate the drive assembly; and
a controller comprising a non-transitory computer-readable medium storing instructions that, when executed, cause the motor to actuate the drive assembly to deliver a stored torque profile to the upper member and the lower member about the rotational axis.

2. The device of claim 1, wherein the drive assembly comprises a capstan drive including a first drum operatively coupled to the motor, a second drum operatively coupled to the rotational axis and the torque cell, and a cord wrapped around the second drum and fixed to opposing ends of the first drum.

3. The device of claim 1, further comprising at least one angle measurement sensor configured to detect angular displacement at either the motor shaft or the output joint.

4. The device of claim 3, further comprising a first angle measurement sensor disposed at the motor shaft and a second angle measurement sensor disposed at the output joint.

5. The device of claim 1, further comprising an interface configured to receive a torque profile and instructions, and store the received torque profile as the stored torque profile.

6. The device of claim 1, wherein the torque cell is configured to measure torque at the rotational axis and transmit the measured torque to the controller.

7. The device of claim 1, wherein the interface is further configured to output stored torque measurements or received torque measurements to an external computing device or display.

8. The device of claim 1, wherein the controller is configured to execute a control algorithm comprising proportional-integral-derivative logic to regulate motor operation based on feedback from the torque cell and angle measurement sensors.

9. The device of claim 1, wherein the controller is configured to adjust motor response in real time based on torque measurements received from the torque cell.

10. The device of claim 9, wherein the controller is further configured to adjust motor response in real time based on a received torque response profile.

11. The device of claim 1, wherein the motor assembly is configured to actuate the drive assembly to produce flexion of the lower member relative to the upper member.

12. The device of claim 1, wherein the motor assembly is configured to actuate the drive assembly to produce extension of the lower member relative to the upper member.

13. A method of using a leg isoinertial measurement brace, the method comprising:
providing a device comprising:
an upper member adapted to fit a wearer's upper leg;
a lower member adapted to fit a wearer's lower leg;
wherein the upper member and the lower member are rotationally coupled about a rotational axis;
a torque cell operatively coupled to the upper member or the lower member and configured to measure torque at the rotational axis;
a drive assembly configured to transmit torque to the upper member and the lower member about the rotational axis;
a motor assembly coupled to the drive assembly to actuate the drive assembly; and
a controller comprising a non-transitory computer-readable medium storing instructions that, when executed, cause the motor to actuate the drive assembly to deliver a stored torque profile to the upper member and the lower member about the rotational axis;
securing the upper member to the wearer's upper leg and the lower member to the wearer's lower leg such that the upper member and lower member are rotationally coupled about the wearer's knee;
operating the controller to actuate the motor assembly and drive assembly to deliver the stored torque profile to the wearer's leg about the rotational axis;
measuring, via the torque cell, torque at the rotational axis during actuation;
transmitting the measured torque to the controller; and
outputting torque measurements or related data to an external computing device or display.

14. A system for isoinertial measurement and control, comprising:
a device comprising:
an upper member adapted to fit a wearer's upper leg;
a lower member adapted to fit a wearer's lower leg;
wherein the upper member and the lower member are rotationally coupled about a rotational axis;
a torque cell operatively coupled to the upper member or the lower member and configured to measure torque at the rotational axis;
a drive assembly configured to transmit torque to the upper member and the lower member about the rotational axis;
a motor assembly coupled to the drive assembly to actuate the drive assembly;
at least one angle measurement sensor configured to detect angular displacement at either a motor shaft or an output joint; and
a controller comprising a non-transitory computer-readable medium storing instructions that, when executed, cause the motor to actuate the drive assembly to deliver a stored torque profile to the upper member and the lower member about the rotational axis; and
a measurement and control system operatively coupled to the device and configured to:
receive a torque profile and instructions via an interface and store the received torque profile as the stored torque profile;
execute a control algorithm comprising proportional-integral-derivative logic to regulate motor operation based on feedback from the torque cell and the at least one angle measurement sensor;
adjust motor response in real time based on measured torque and a received torque response profile; and
output torque measurements or related data to an external computing device or display.
